(19) United States
(12) Patent Application Publication
(10) Pub. No.: US 2024/0016440 A1
(43) Pub. Date: Jan. 18, 2024

(54) DIAGNOSIS AND TREATMENT DETERMINATION USING NON-CONTACT MONITORING

(71) Applicant: Covidien LP, Mansfield, MA (US)

(72) Inventors: Thomas I. MILLER, Blaine, MN (US); Sajith ANANTHARAMAN, Maple Grove, MN (US); Paul S. ADDISON, Edinburgh (GB); Robert T. SANDGREN, Lindstrom, MN (US); Jeffrey P. BODNER, Plymouth, MN (US); Adam J. RIVARD, Blaine, MN (US); Avram SCHEINER, Vadnais Heights, MN (US); Ryan B. SEFKOW, Plymouth, MN (US); Ohad COHEN, Kiryat Uno (IL)

(21) Appl. No.: 18/352,598

(22) Filed: Jul. 14, 2023

Related U.S. Application Data
(60) Provisional application No. 63/368,569, filed on Jul. 15, 2022.
(60) Provisional application No. 63/369,887, filed on Jul. 29, 2022.

(51) Int. Cl. A61B 5/00; A61B 5/11; G16H 50/20; G16H 20/40; A61N 1/05

(52) U.S. Cl. CPC A61B 5/4082; A61B 5/1101; A61B 5/1126; A61B 5/742; G16H 50/20; G16H 20/40; A61N 1/0529

(57) ABSTRACT

Use of non-contact patient monitoring systems to detect, diagnose, monitor and/or adjust therapy for diseases or conditions that have motion-based symptoms, symptoms such as limb tremors, spasms, etc. that can be correlated to diseases or conditions such as Essential tremor, Parkinson's disease, restless leg syndrome, or a diabetic episode. Information garnered by the non-contact monitoring system, information such as location, frequency, severity, and duration of the motion, can be collected and analyzed so that a diagnosis can be made and/or a treatment plan developed and/or adjusted by the patient's caretaker. The non-contact monitoring can provide real-time feedback regarding effectiveness of therapies or treatments, such as, e.g., neurological stimulation.

### CROSS-REFERENCE

This application claims benefit of priority to U.S. provisional application Ser. No. 63/368,569 filed Jul. 15, 2022, and titled “Diagnosis and Treatment Determination Using Non-Contact Monitoring,” and to U.S. provisional application Ser. No. 63/369,887 filed Jul. 29, 2022, and titled “Monitoring for Diabetic Events Using Non-Contact Monitoring System,” the entire disclosures of which are incorporated herein by reference for all purposes.

### BACKGROUND

Many conventional medical monitors require attachment of a sensor to a patient in order to detect physiologic signals from the patient and transmit detected signals through a cable to the monitor. These monitors process the received signals and determine vital signs such as the patient's pulse rate, respiration rate, and arterial oxygen saturation.

Other monitoring systems include other types of monitors and sensors, such as electroencephalogram (EEG) sensors, blood pressure cuffs, temperature probes, air flow measurement devices (e.g., spirometer), and others. Some wireless, wearable sensors have been developed, such as wireless EEG patches and wireless pulse oximetry sensors.

Video-based monitoring is a new field of patient monitoring that uses a remote video camera to detect physical attributes of the patient. This type of monitoring may also be called “non-contact” monitoring in reference to the remote video sensor, which does not contact the patient.

### SUMMARY

The present disclosure is directed to using non-contact patient monitoring systems to detect, diagnose, monitor and/or adjust therapy for diseases and conditions that have motion-based symptoms, symptoms such as limb tremors, spasms, etc. Information garnered by the non-contact monitoring system, information such as location, frequency, severity, and duration of the motion, can be collected and analyzed so that a diagnosis can be made and/or a treatment plan developed and/or adjusted by the patient's caretaker. Additionally, the non-contact monitoring can provide real-time feedback regarding effectiveness of therapies or treatments, such as, e.g., neurological stimulation. Real-time feedback from the non-contact system can provide quantitative data to the caretaker to aid in therapy adjustment.

One particular embodiment described herein is a method of treating a patient, the method comprising detecting movement of a patient in a region of interest (ROI) with a non-contact monitoring system, correlating the detected movement to a disease or condition, and determining a treatment for the patient for the disease or condition. The detected movement may be correlated to a disease or condition such as restless leg syndrome, Essential tremor, or Parkinson's disease.

Another particular embodiment described herein is a method of treating a patient, the method comprising detecting movement of a patient in a region of interest (ROI) with a non-contact monitoring system, providing a therapy to the patient based on the detected movement, after providing the therapy, detecting modified movement of the patient in the ROI with the non-contact monitoring system, and adjusting the therapy to the patient based on the detected modified movement.

Yet another particular embodiment described herein is a non-contact monitoring system for monitoring a patient. The system includes a depth-sensing camera, a display, and a processor operably connected to a memory of the system. The processor is configured to implement steps to detect movement of the patient in a region of interest (ROI) with the non-contact monitoring system based on a change in a depth signal over time, correlate the detected movement to a disease or condition, and determine a treatment for the patient for the disease or condition.

Other embodiments are also described and recited herein.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWING

FIG. 1 is a schematic diagram of an example non-contact patient monitoring system.

FIG. 2 is a schematic diagram of another example non-contact patient monitoring system.

FIG. 3A and FIG. 3B are schematic diagrams showing two embodiments using the example non-contact patient monitoring system of FIG. 2.

FIG. 4 is a block diagram of a computing device, a server, and an image capture device according to various embodiments described herein.

FIG. 5 is a schematic diagram of a non-contact patient monitoring system monitoring a patient in a first region.

FIG. 6 is a schematic diagram of a non-contact patient monitoring system monitoring a patient in a first and second region.

FIG. 7 is a schematic diagram of a non-contact patient monitoring system monitoring a patient in a third region.

FIG. 8 is a stepwise method of an example method of determining treatment for a disease with motion-based symptoms according to various embodiments described herein.

FIG. 9 is a stepwise method of an example method of determining and adjusting treatment for a disease with motion-based symptoms according to various embodiments described herein.

FIG. 10 is a stepwise method of an example method of determining a diabetic event.

FIG. 11 is a stepwise method of an example method of determining a diabetic event and providing treatment therefor.

### DETAILED DESCRIPTION

As described above, the present disclosure is directed to monitoring for a medical diagnosis and treatment options therefore, and in particular, non-contact, video-based monitoring of motion or movement to diagnose and evaluate certain diseases and conditions that have motion-based symptoms. Systems and methods are described for receiving a video signal view of a patient, identifying a physiologically relevant area within the video image (such as a patient's legs, arms, or torso), extracting a distance or depth signal from the relevant area, correlating the changing depth signals over time to motion, and using that indication of motion to diagnose and determine a treatment for the patient.

The non-contact monitoring systems can be used to monitor whether or not a particular condition or symptom is present, and if detected, can aid in diagnosing the patient and/or providing a treatment to the patient. The monitoring may be done during daytime or nighttime, typically when the patient is resting, sleeping, or inactive. Examples of diseases and conditions that have motion-based symptoms include diabetes, restless leg syndrome, dyskinesia, Parkinson's disease, and Essential tremor. A diabetic episode often includes restlessness and/or increased respiratory rate. Additionally, the non-contact monitoring systems can determine and accurately record the occurrence (e.g., frequency, amplitude, duration, etc.) of movement symptoms.

As an example for diabetes, the non-contact monitoring systems can detect, e.g., hyperventilation or increased respiratory rate, which could be caused by the accumulation of blood ketones, which can be indicative of diabetic ketoacidosis (DKA) associated with hyperglycemia. The non-contact monitoring systems can also detect, e.g., increasing overall movement or unrest (e.g., hypermobility), which is indicative of hypoglycemia.

Hyperglycemia is when there is too much sugar (glucose) in the blood, typically because of a low level or lack of insulin. Hyperglycemia can cause vomiting, excessive hunger and/or thirst, rapid heartbeat, and shortness of breath. Hyperventilating or an increased respiratory rate is a common, readily noticeable symptom of hyperglycemia, as the body attempts to expel ketones, which are metabolites of fatty acids, which result due to the excess blood sugar. To treat hyperglycemia, insulin is administered to the body.

The opposite, hypoglycemia, is when there is a too low level of sugar (glycose) in the blood. Hypoglycemia can cause headache, sweating, fatigue, an irregular or fast heartbeat, shakiness, and overall unrest. To treat hypoglycemia, either sugar or medication is administered to the body; sugar can be readily administered by foods such as honey, chocolate or candy, or sugared soft drinks.

For both hyperglycemia and hypoglycemia, movement (motion) of the patient can be monitored by non-contact monitoring systems to determine, for example, if the patient is hyperventilating or has increased respiratory rate, or if the patient has overall increased unrest, often indicated by progressively increased movement. Additionally, the non-contact monitoring systems can detect an irregular or increased heartbeat.

Shaking or trembling hands are symptoms for both Parkinson's disease and Essential tremor, both which can be monitored by the systems described herein. However, the treatments for the two are different; thus knowing which disease is present is paramount. Non-contact monitoring of the movement can accurately determine the type and frequency of the movement, thus leading to an accurate diagnosis.

Both Parkinson's disease and Essential tremor result in shaking of the patient's hands. Essential tremor of the hands usually occurs when the patient uses their hands, for example, while eating, tying shoes, brushing teeth, etc. Essential tremor mainly involves the hands but can also involve the patient's head and voice. Tremors from Parkinson's disease are most prominent when the patient's hands are idle, hanging at the patient's sides or resting in their lap. Although Parkinson's disease tremors usually start in the patient's hands, the tremors can affect the legs, chin and other parts of the patient's body. Knowing which portions of the body are experiencing tremors can aid in the diagnosis.

Restless leg syndrome (RLS), also known as Willis-Ekorn disease, causes an uncontrollable urge for the patient to move their legs, usually because of an uncomfortable sensation. In some instances, the patient has uncontrolled movement of the legs, such as spasms. RLS is typically most noticed in the evening or nighttime hours when the patient is sitting or lying down.

Dyskinesia is a condition that produces involuntary, erratic, writhing movements of the face, arms, legs and/or torso. The muscle spasms may be fluid and slow or may be rapid, causing jerking. Dyskinesia may be caused by medication or a brain injury such as a vascular event (e.g., stroke, aneurysm).

Non-contact monitoring systems can be used to detect the motions or movements, identify the type of motion or movement, and facilitate an accurate diagnosis.

Signals representative of the movement or motion of the patient are detected by a depth sensing camera or camera system that views but does not contact the patient. With appropriate selection and filtering of the signals detected by the camera, the physiologic contribution by the detected depth signal can be isolated and measured. Additionally, with appropriate selection and filtering of the signal detected, the physiologic contribution by a light intensity signal can be estimated or calculated. This approach has the potential to improve patient diagnosis and ongoing treatment for various diseases that have a motion-based symptoms component.

Remote sensing of a patient with video-based monitoring systems presents several challenges. One challenge is ambient light. In this context, “ambient light” means surrounding light not emitted by components of the camera or the monitoring system. In some embodiments of the non-contact monitoring systems, the desired physiologic signal is generated or carried by a light source. Thus, because of this, the ambient light cannot be entirely filtered, removed, or avoided as noise. Changes in lighting within the room, including overhead lighting, sunlight, television screens, variations in reflected light, and passing shadows from moving objects all contribute to the light signal that reaches the camera. Even subtle motions outside the field of view of the camera can reflect light onto the patient being monitored.

Non-contact monitoring such as video-based monitoring can deliver significant benefits over contact monitoring. Some video-based monitoring systems and methods can reduce cost and waste by reducing use of disposable contact sensors, replacing them with reusable camera systems. Video monitoring may also reduce the spread of infection, by reducing physical contact between caregivers and patients. Video cameras can improve patient mobility and comfort, by freeing patients from wired tethers or bulky wearable sensors. In some cases, these systems can also save time for caregivers, who no longer need to reposition, clean, inspect, or replace contact sensors. Additionally, video cameras provide evidential documentation.

The present disclosure describes methods of non-contact monitoring of a patient to determine an identification, diagnosis and possible treatment therefor of certain diseases having a motion-based symptoms component. The monitoring may be done in daylight or in a lighted environment, or at night, in a dark environment. The patient may be, e.g., laying down or sitting.

The non-contact monitoring systems receive a video signal from the patient and from that extract a distance or depth signal from the relevant area to calculate the movement or motion from the depth signal. The systems can also receive a second signal, a light intensity signal reflected from the patient, and from that calculate the movement or motion from the light intensity signal. The movement or motion parameters from the two signals can be combined or compared to provide a qualified output parameter. In some embodiments, the light intensity signal is a reflection of an IR feature projected onto the patient, such as by a projector.

The depth sensing feature of the systems provides a measurement of the distance or depth between the detection system and the patient. One or two video cameras may be used to determine the depth, and change in depth, from the camera to the patient. When two cameras, set at a fixed distance apart, are used, they offer stereo vision due to the slightly different perspectives of the scene from which distance information is extracted. When distinct features are present in the scene, the stereo image algorithm can find the locations of the same features in the two image streams. However, if an object is featureless (e.g., a smooth surface with a monochromatic color), then the depth camera system may have difficulty resolving the perspective differences. By including an image projector to project features (e.g., in the form of dots, pixels, etc.) onto the scene, this projected feature can be monitored over time to produce an estimate of changing distance or depth.

In the following description, reference is made to the accompanying drawing that forms a part hereof and in which is shown by way of illustration at least one specific embodiment. The following description provides additional specific embodiments. It is to be understood that other embodiments are contemplated and may be made without departing from the scope or spirit of the present disclosure. The following detailed description, therefore, is not to be taken in a limiting sense. While the present disclosure is not so limited, an appreciation of various aspects of the disclosure will be gained through a discussion of the examples, including the figures, provided below. In some instances, a reference numeral may have an associated sub-label consisting of a lower-case letter to denote one of multiple similar components. When reference is made to a reference numeral without specification of a sub-label, the reference is intended to refer to all such multiple similar components.

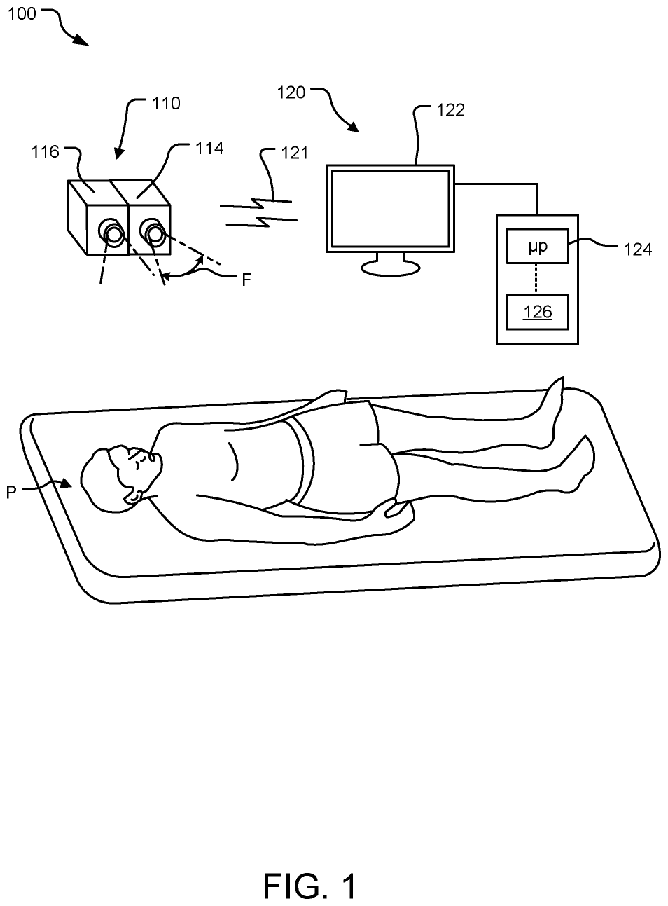

FIG. 1 shows a non-contact patient monitoring system 100 and a patient P. The system 100 includes a non-contact detector system 110 placed remote from the patient P. In this embodiment, the detector system 110 includes a camera system 114, particularly, a camera that includes an infrared (IR) detection feature. The camera 114 may be a depth sensing camera, such as a Kinect camera from Microsoft Corp. (Redmond, Washington) or a Real Sense™ D415, D435 or D455 camera from Intel Corp. (Santa Clara, California). The camera system 114 is remote from the patient P, in that it is spaced apart from and does not physically contact the patient P. The camera system 114 may be, for example, mounted on a stand (e.g., a rollable stand) or affixed to a wall proximate the patient P, or to the bed of the patient P. The camera system 114 includes a detector exposed to a field of view F that encompasses at least a portion of the patient P.

The camera system 114 includes a depth sensing camera that can detect a distance between the camera system 114 and objects in its field of view F. Such information can be used, as disclosed herein, to determine that a patient is within the field of view of the camera system 114 and determine a region of interest (ROI) to monitor on the patient. Once an ROI is identified, that ROI can be monitored over time, and the change in depth of points within the ROI can represent movements of the patient associated with, e.g., tremors, or hyperventilation.

The field of view F is selected based on the movement being monitored. For example, for a patient with possible restless leg syndrome, the legs of the patient can be within the field of view. As another example, for a patient with possible tremors (e.g., Parkinson's disease, Essential tremor, dyskinesia), the field of view F can be on the hands/arms of the patient or on the head and face. For a patient with a known tendency for hyperglycemia, the chest of the patient can be within the field of view, to monitor respiration rate and/or volume. As another example, for a patient with a known tendency for hypoglycemia, the field of view F can be on the hands/arms of the patient or on the legs, to monitor for overall restless movement.

In some embodiments, the field of view F encompasses exposed skin of the patient. In other embodiments, the field of view F encompasses a monitored portion of the patient as covered by a blanket, sheet, or gown.

The camera system 114 operates at a frame rate, which is the number of image frames taken per second (or other time period). Example frame rates include 20, 30, 40, 50, or 60 frames per second, greater than 60 frames per second, or other values between those. Frame rates of 20-30 frames per second produce useful signals, though frame rates above 100 or 120 frames per second are helpful in avoiding aliasing with light flicker (for artificial lights having frequencies around 50 or 60 Hz).

The distance from the ROI on the patient P to the camera system 114 is measured by the system 100. Generally, the camera system 114 detects a distance between the camera system 114 and the surface within the ROI; the change in depth or distance of the ROI can represent movements of the patient, e.g., tremors, leg spasms, chest movement due to breathing.

In some embodiments, the system 100 determines a skeleton outline of the patient P to identify a point or points from which to extrapolate the ROI. For example, a skeleton may be used to find a center point of a chest, shoulder points, waist points, hands, feet or knees, and/or any other points on a body. These points can be used to determine the ROI. For example, the ROI may be defined by filling in the area around the knees. Certain determined points may define an outer edge of an ROI, such as waist points. In other embodiments, instead of using a skeleton, other points are used to establish an ROI. For example, a face may be recognized, and a torso and waist area inferred in proportion and spatial relation to the face. In other embodiments, the system 100 may establish the ROI around a point based on which parts are within a certain depth range of the point. In other words, once a point is determined that an ROI should be developed from, the system can utilize the depth information from the depth sensing camera system 114 to fill out the ROI as disclosed herein. For example, if a point on the chest is selected, depth information is utilized to determine the ROI area around the determined point that is a similar distance from the depth sensing camera 114 as the determined point. This area is likely to be a chest.

In another example, the patient P may wear a specially configured piece of clothing that identifies points on the body such as the legs or the hands. The system 100 may identify those points by identifying the indicating feature of the clothing. Such identifying features could be a visually encoded message (e.g., bar code, QR code, etc.), or a brightly colored shape that contrasts with the rest of the patient's clothing, etc. In some embodiments, a piece of clothing worn by the patient may have a grid or other identifiable pattern on it to aid in recognition of the patient and/or their movement. In some embodiments, the identifying feature may be stuck on the clothing using a fastening mechanism such as adhesive, a pin, etc., or stuck directly on the patient's skin, such as by adhesive. For example, a small sticker or other indicator may be placed on a patient's hands that can be easily identified from an image captured by a camera. In some embodiments, the indicator may be a sensor that can transmit a light or other information to the camera system 114 that enables its location to be identified in an image so as to help define the ROI. Therefore, different methods can be used to identify the patient and define an ROI.

The ROI size may differ according to the distance of the patient from the camera system. The ROI dimensions may vary linearly with the distance of the patient from the camera system. This ensures that the ROI scales according with the patient and covers the same part of the patient regardless of the patient's distance from the camera. This is accomplished by applying a scaling factor that is dependent on the distance of the patient (and the ROI) from the camera. In order to properly measure the depth changes, the actual size (area) of the ROI is determined and movements of that ROI are measured. The measured movements of the ROI and the actual size of the ROI are then used to calculate the respiratory parameter, e.g., a tidal volume. Because a patient's distance from a camera can change, e.g., due to rolling or position readjustment, the ROI associated with that patient can appear to change in size in an image from a camera. However, using the depth sensing information captured by a depth sensing camera or other type of depth sensor, the system can determine how far away from the camera the patient (and their ROI) actually is. With this information, the actual size of the ROI can be determined, allowing for accurate measurements of depth change regardless of the distance of the camera to the patient.

In some embodiments, the system 100 may receive a user input to identify a starting point for defining an ROI. For example, an image may be reproduced on an interface, allowing a user of the interface to select a point on the patient from which the ROI can be determined (such as a point on the legs). Other methods for identifying a patient, points on the patient, and defining an ROI may also be used.

However, if the ROI is essentially featureless (e.g., a smooth surface with a monochromatic color, such as a blanket or sheet covering the patient P), then the camera system 114 may have difficulty resolving the perspective differences. To address this, the system 100 includes a projector 116 to project individual features (e.g., dots, crosses or Xs, lines, individual pixels, etc.) onto the ROI; the features may be visible light, UV light, infrared (IR) light, etc. The projector may be part of the detector system 110 or the overall system 100.

The projector 116 generates a sequence of features over time on the ROI from which is monitored and measured the reflected light intensity. A measure of the amount, color, or brightness of light within all or a portion of the reflected feature over time is referred to as a light intensity signal. The camera system 114 detects the features from which this light intensity signal is determined. In an embodiment, each visible image projected by the projector 116 includes a two-dimensional array or grid of pixels, and each pixel may include three color components—for example, red, green, and blue. A measure of one or more color components of one or more pixels over time is referred to as a “pixel signal,” which is a type of light intensity signal. In another embodiment, when the projector 116 projects an IR feature, which is not visible to a human eye, the camera system 114 includes an infrared (IR) sensing feature. In another embodiment, the projector 116 projects a UV feature. In yet other embodiments, other modalities including millimeter-wave, hyper-spectral, etc., may be used.

The projector 116 may alternately or additionally project a featureless intensity pattern (e.g., a homogeneous, a gradient or any other pattern that does not necessarily have distinct features). In some embodiments, the projector 116, or more than one projector, can project a combination of a feature-rich pattern and featureless patterns on to the ROI.

The light intensity of the image reflected by the patient surface is detected by the detector system 110.

The detected images and/or diffusion measurements are sent to a computing device 120 through a wired or wireless connection 121. The computing device 120 includes a display 122, a processor 124, and hardware memory 126 for storing software and computer instructions. Sequential image frames of the patient P are recorded by the video camera system 114 and sent to the computing device 120 for analysis by the processor 124. The display 122 may be remote from the computing device 120, such as a video screen positioned separately from the processor and memory. Other embodiments of the computing device 120 may have different, fewer, or additional components than shown in FIG. 1. In some embodiments, the computing device may be a server. In other embodiments, the computing device of FIG. 1 may be connected to a server. The captured images (e.g., still images or video) can be processed or analyzed at the computing device and/or at the server to determine the motion of the patient P as disclosed herein.

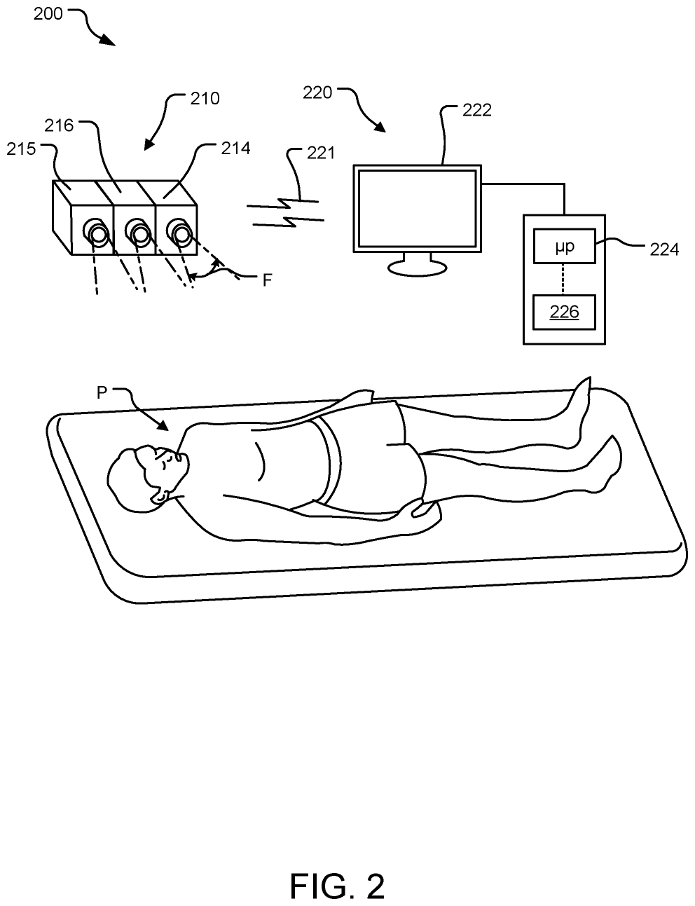

FIG. 2 shows another non-contact patient monitoring system 200 and a patient P. The system 200 includes a non-contact detector 210 placed remote from the patient P. In this embodiment, the detector 210 includes a first camera 214 and a second camera 215, at least one of which includes an infrared (IR) camera feature. The cameras 214, 215 are positioned so that their ROIs at least intersect, in some embodiments, completely overlap. The detector 210 also includes an IR projector 216, which projects individual features (e.g., dots, crosses or Xs, lines, or a featureless pattern, or a combination thereof etc.) onto the ROI. The projector 216 can be separate from the detector 210 or integral with the detector 210, as shown in FIG. 2. In some embodiments, more than one projector 216 can be used. Both cameras 214, 215 are aimed to have features projected by the projector 216 to be in their ROI. The cameras 214, 215 and projector 216 are remote from the patient P, in that they are spaced apart from and do not contact the patient P. In this implementation, the projector 216 is physically positioned between the cameras 214, 215, whereas in other embodiments it may not be so.

The distance from the ROI to the cameras 214, 215 is measured by the system 200. Generally, the cameras 214, 215 detect a distance between the cameras 214, 215 and the projected features on a surface within the ROI. The light from the projector 216 hitting the surface is scattered/diffused in all directions; the diffusion pattern depends on the reflective and scattering properties of the surface. The cameras 214, 215 also detect the light intensity of the projected individual features in their ROIs. From the distance and the light intensity, movement of the patient P is monitored.

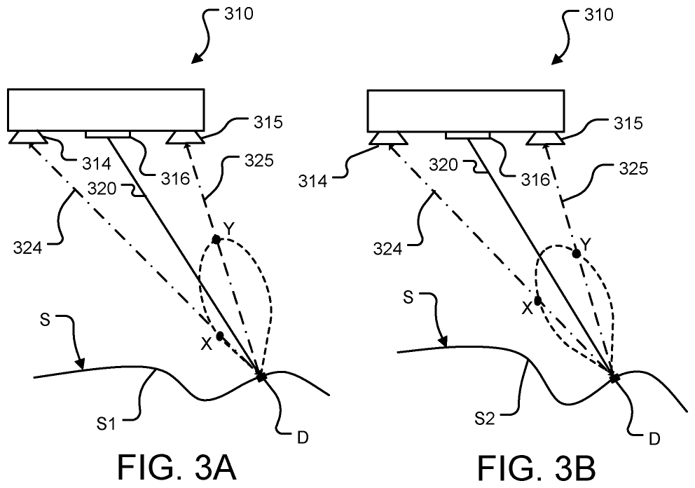

FIG. 3A and FIG. 3B both show a non-contact detector 310 having a first camera including an IR detection feature 314, a second IR camera including an IR detection feature 315, and an IR projector 316. A dot D is projected by the projector 316 onto a surface S, e.g., of a patient, via a beam 320. Light from the dot D is reflected by the surface S and is detected by the camera 314 as beam 324 and by the camera 315 as beam 325.

The light intensity returned to and observed by the cameras 314, 315 depends on the diffusion pattern caused by the surface S (e.g., the surface of a patient), the distance between the cameras 314, 315 and surface S, the surface gradient, and the orientation of the cameras 314, 315 relative to the surface S. In FIG. 3A, the surface S has a first profile S1 and in FIG. 3B, the surface S has a second profile S2 different than S1; as an example, the first profile S1 is with the patient in a first position and the second profile S2 is with the patient in a second position. Because the surface profiles S1 and S2 differ, the deflection pattern from the dot D on each of the surfaces differs for the two figures.

During movement of the patient, the light intensity reflection off the dot D observed by the cameras 314, 315 changes because the surface profile S1 and S2 (specifically, the gradient) changes as well as the distance between the surface S and the cameras 314, 315. FIG. 3A shows the surface S having the surface profile S1 at time instant t=tₙ and FIG. 3B shows the surface S having the surface profile S2 at a later time, specifically t=tₙ₊₁, with S2 being slightly changed due to movement. Consequently, the intensity of the projected dot D observed by the cameras 314, 315 will change due to the changes of the surface S. In FIG. 3A, a significantly greater intensity is measured by the camera 315 than by the camera 314, seen by the x and y on the beams 324, 325, respectively. In FIG. 3B, y is less than y in FIG. 3A, whereas x in FIG. 3B is greater than x in FIG. 3A. The manner in how these intensities change depends on the diffusion pattern and its change over time. As seen in FIGS. 3A and 3B, the light intensities as measured by the cameras 314 and 315 have changed between FIGS. 3A and 3B, and hence, the surface S has moved. Each camera will generate a signal because of the change of the intensity of dot D when the surface profile changes from time instant t=tₙ to t=tₙ₊₁ due to movement.

In some other embodiments, a single camera and light projector can be used. For example, in FIGS. 3A and 3B, the camera 315 is not present or is ignored; the camera 314 will still produce a change in light intensity from time instant t=tₙ to t=tₙ₊₁ due to movement. This embodiment will therefore produce only a single signal as opposed to the two signals generated by the embodiment discussed in the previous paragraph.

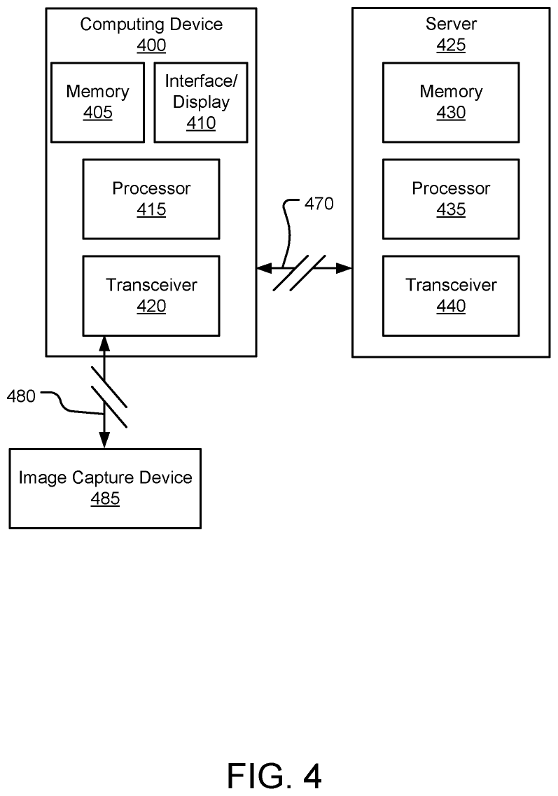

FIG. 4 is a block diagram illustrating a system including a computing device 400, a server 425, and an image capture device 485 (e.g., a camera, e.g., the camera system 114 or cameras 214, 215). In various embodiments, fewer, additional and/or different components may be used in the system.

The computing device 400 includes a processor 415 that is coupled to a memory 405. The processor 415 can store and recall data and applications in the memory 405, including applications that process information and send commands/signals according to any of the methods disclosed herein. The processor 415 may also display objects, applications, data, etc. on an interface/display 410. The processor 415 may also or alternately receive inputs through the interface/display 410. The processor 415 is also coupled to a transceiver 420. With this configuration, the processor 415, and subsequently the computing device 400, can communicate with other devices, such as the server 425 through a connection 470 and the image capture device 485 through a connection 480. For example, the computing device 400 may send to the server 425 information determined about a patient from images captured by the image capture device 485, such as depth information of a patient in an image.

The server 425 also includes a processor 435 that is coupled to a memory 430 and to a transceiver 440. The processor 435 can store and recall data and applications in the memory 430. With this configuration, the processor 435, and subsequently the server 425, can communicate with other devices, such as the computing device 400 through the connection 470.

The computing device 400 may be, e.g., the computing device 120 of FIG. 1 or the computing device 220 of FIG. 2. Accordingly, the computing device 400 may be located remotely from the image capture device 485, or it may be local and close to the image capture device 485 (e.g., in the same room). The processor 415 of the computing device 400 may perform any or all of the various steps disclosed herein. In other embodiments, the steps may be performed on a processor 435 of the server 425. In some embodiments, the various steps and methods disclosed herein may be performed by both of the processors 415 and 435. In some embodiments, certain steps may be performed by the processor 415 while others are performed by the processor 435. In some embodiments, information determined by the processor 415 may be sent to the server 425 for storage and/or further processing.

The devices shown in the illustrative embodiment may be utilized in various ways. For example, either or both of the connections 470, 480 may be varied. For example, either or both the connections 470, 480 may be a hard-wired connection. A hard-wired connection may involve connecting the devices through a USB (universal serial bus) port, serial port, parallel port, or other type of wired connection to facilitate the transfer of data and information between a processor of a device and a second processor of a second device. In another example, one or both of the connections 470, 480 may be a dock where one device may plug into another device. As another example, one or both of the connections 470, 480 may be a wireless connection. These connections may be any sort of wireless connection, including, but not limited to, Bluetooth connectivity, Wi-Fi connectivity, infrared, visible light, radio frequency (RF) signals, or other wireless protocols/methods. For example, other possible modes of wireless communication may include near-field communications, such as passive radio-frequency identification (RFID) and active RFID technologies. RFID and similar near-field communications may allow the various devices to communicate in short range when they are placed proximate to one another. In yet another example, the various devices may connect through an internet (or other network) connection. That is, one or both of the connections 470, 480 may represent several different computing devices and network components that allow the various devices to communicate through the internet, either through a hard-wired or wireless connection. One or both of the connections 470, 480 may also be a combination of several modes of connection.

The configuration of the devices in FIG. 4 is merely one physical system on which the disclosed embodiments may be executed. Other configurations of the devices shown may exist to practice the disclosed embodiments. Further, configurations of additional or fewer devices than the ones shown in FIG. 4 may exist to practice the disclosed embodiments. Additionally, the devices shown in FIG. 4 may be combined to allow for fewer devices than shown or separated such that more than the three devices exist in a system. It will be appreciated that many various combinations of computing devices may execute the methods and systems disclosed herein. Examples of such computing devices may include other types of medical devices and sensors, infrared cameras/detectors, night vision cameras/detectors, other types of cameras, radio frequency transmitters/receivers, smart phones, personal computers, servers, laptop computers, tablets, RFID enabled devices, or any combinations of such devices.

The method of this disclosure utilizes depth (distance) information between the camera(s) and the patient to determine movement, e.g., repeated movement. A depth image or depth map, which includes information about the distance from the camera to each point in the image, can be measured or otherwise captured by a depth sensing camera, such as a Kinect camera from Microsoft Corp. (Redmond, Washington) or a Real Sense™ D415, D435 or D455 camera from Intel Corp. (Santa Clara, California) or other sensor devices based upon, for example, millimeter wave and acoustic principles to measure distance.

The depth image or map can be obtained by a stereo camera, a camera cluster, camera array, or a motion sensor focused on a ROI, such as a patient's hands or legs. In some embodiments, the camera(s) are focused on visible or IR features in the ROI. Each projected feature may be monitored, less than all the features in the ROI may be monitored or all the pixels in the ROI can be monitored.

When multiple depth images are taken over time in a video stream, the video information includes the movement of the points within the image, as they move toward and away from the camera over time.

Because the image or map includes depth data from the depth sensing camera, information on the spatial location of the patient (e.g., the patient's legs) in the ROI can be determined. This information can be contained, e.g., within a matrix. As the patient's legs move, e.g., spasm, the patient's legs move toward and away from the camera, changing the depth information associated with the images over time. As a result, the location information associated with the ROI changes over time.

As indicated above, in addition to the methodology of this disclosure utilizing depth (distance) information between the camera(s) and the patient to determine movement of the patient, e.g., the patient's hands, legs, feet; the method can also use reflected light intensity from projected IR features (e.g., dots, grid, stripes, crosses, squares, etc., or a featureless pattern, or a combination thereof) in the scene to estimate the depth (distance).

This change of intensity of each of the projected features is used to indicate movement of the surface on which the feature was projected. The intensity signal is formed by aggregating all the pixel values, at an instant in time, from across the ROI to generate a pattern signal. In some embodiments, less than all the projected features in the ROI are monitored; for example, only a random sampling of the projected features is monitored, or for example, every third feature is monitored. In some embodiments, each feature reflection is monitored only for a predetermined duration, to determine which projected features provide an accurate or otherwise desired light intensity signal, and then those selected features are monitored to obtain the signal. In some embodiments, each pixel in the ROI is monitored and the light intensity signal obtained.

This method for producing a movement signal, i.e., from the intensity of the light diffusion, is independent from the depth data used to produce a signal representative of the movement. This secondary pattern signal, from the light intensity, can be used to enhance or confirm the measurement from the depth data. The movement indicated by the varying reflected intensity, typically closely matches the movement determined by the depth (distance) measured by the depth camera(s), e.g., camera system 114, cameras 214, 215.

Returning to and with respect to FIG. 2 and FIGS. 3A and 3B above, it is described that the system 200 with two cameras 214, 215 or the system with two cameras 314, 315 can be used, the two cameras 214, 215 and 314, 315 providing a stereo property for one or both of the depth signal and the light intensity signal. When two cameras are used, although both cameras will produce very similar results, they each have their own noise characteristics. The noise, which is added to the movement signal, is generally uncorrelated and the overall noise component is therefore reduced by combining the results of two cameras. Thus, each camera produces a movement pattern and the results may then be, for example, averaged. Note that more than two cameras may be used to further improve the performance. Additionally, e.g., other, more advanced, methods for combining/fusing the different signals may be used including Kalman and particle filtering.

Thus, described herein are methods and systems for non-contact monitoring of a patient to determine movement by utilizing a distance or depth signal from the patient to the system and by utilizing a reflected light intensity signal from projected IR features to derive the same parameter(s). The parameter(s) from the two signals are combined or compared to provide an output parameter value or signal.

These methods and systems of non-contact patient monitoring can be used to diagnose and/or treat various diseases or conditions having motion-based symptoms, such as restless leg syndrome, Essential tremor, and Parkinson's disease. The methods and systems can be used to monitor for on-coming diabetic episodes. The non-contact monitoring systems can provide real-time feedback to therapy applied to the patient.

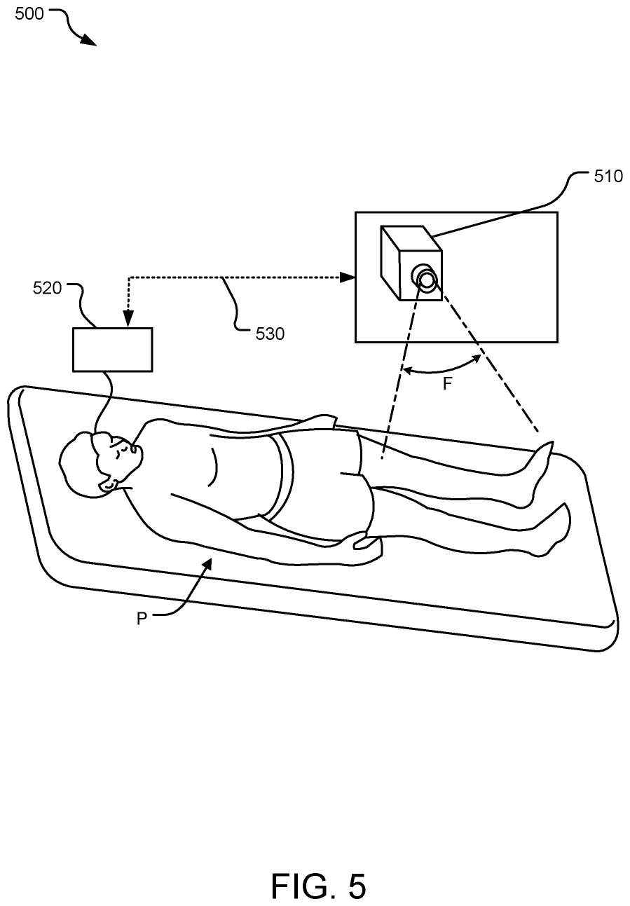

FIG. 5 shows a scenario where a patient P is being monitored by a non-contact system 500 for leg movement, for example, indicative of restless leg syndrome or overall restlessness, a symptom of hypoglycemia. The patient P, particularly the legs in the ROI region, can be monitored for not only movement, but also an increase in movement over time.

The system 500 may be any of the systems 100, 200, or other described above, or variations thereof. The system 500 has a camera system 510 with a field of view F that corresponds to an ROI on the patient P's legs. The camera system 510 can be any camera system (e.g., the camera system 114 or the cameras 214, 215). The camera system 510 monitors the ROI and the system 500 determines and records a signal or signal pattern, as a waveform, that can be used to identify restless leg syndrome events or cycles.

In the particular scenario shown in FIG. 5, the patient P has an implanted neurostimulus device 520, which provides an electrical stimulus, such as a pulse, to a particular area of the patient P (e.g., to a particular nerve, or a portion of the brain). In theory, for restless leg syndrome, a change in the output from the neurostimulus device 520 changes the movement of the legs.

The patient P, particularly the legs in the ROI region, can be monitored for a reduction in severity of or cessation of movement as the output from the neurostimulus device 520 is adjusted. A feedback loop 530 operably connects the neurostimulus device 520 and the system 500, allowing real-time adjustment of the output of the neurostimulus device 520 based on the movement detected by the system 500. This output adjustment may be manual (e.g., by the patient's physician) or may be by software in the neurostimulus device 520 (e.g., the device 520 is programmed to adjust its output).

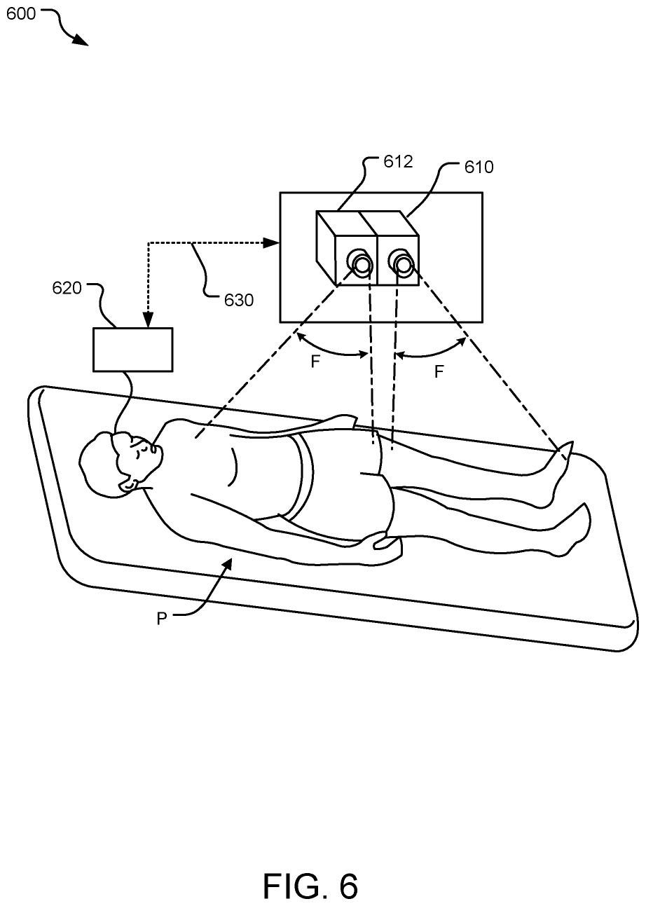

FIG. 6 shows another scenario where a patient P is being monitored by a non-contact system 600. In this scenario, the patient P is generally being monitored for possible movements, or tremors or convulsions, indicative or any of, for example, of restless leg syndrome, Parkinson's disease, Essential tremor, and overall restlessness. This is similar to the scenario of FIG. 5, however in FIG. 6 the ROI is larger, monitoring both the legs and the torso of the patient P.

The system 600 can be used to diagnose a patient P's ailments (e.g., determine what, if any, disease or condition the patient P may have) or to monitor progression of a known disease or condition, or to monitor the patient P's response to therapy or treatment. The system 600 may be any of the systems 100, 200, or other described above, or variations thereof. The system 600 has a first camera system 610 that has a field of view F that corresponds to an ROI on the patient P's legs and a second camera system 612 that has another field of view F that corresponds to an ROI on the patient P's torso. The camera system 610, 612 can be any camera system (e.g., the camera system 114 or the cameras 214, 215).

The camera systems 610, 612 monitor the ROIs and the system 600 determines and records a signal or signal pattern, as a waveform, that can be used to identify movement in the leg area and movement in the torso region. Movement in only or predominantly in the leg area may indicate that the patient P has restless leg syndrome whereas movement in both the leg area and the torso area may indicate that the patient P has a tremor disease or has overall restlessness.

The patient P has an implanted neurostimulus device 620, which provides an electrical stimulus, such as a pulse, to a particular area of the patient P (e.g., to a particular nerve, or a portion of the brain). In theory, depending on the disease or condition of the patient P, a change in the output from the neurostimulus device 620 affects the movement of various limbs of the patient P.

Both the legs in the ROI region and the torso in the ROI region can be monitored for a reduction in severity of or cessation of movement as the output from the neurostimulus device 620 is adjusted. A feedback loop 630 operably connects the neurostimulus device 620 and the system 600, allowing real-time adjustment of the output of the neurostimulus device 620 based on the movement detected by the system 600. This output adjustment may be manual (e.g., by the patient's physician) or may be by software in the neurostimulus device 620 (e.g., the device 620 is programmed to adjust its output).

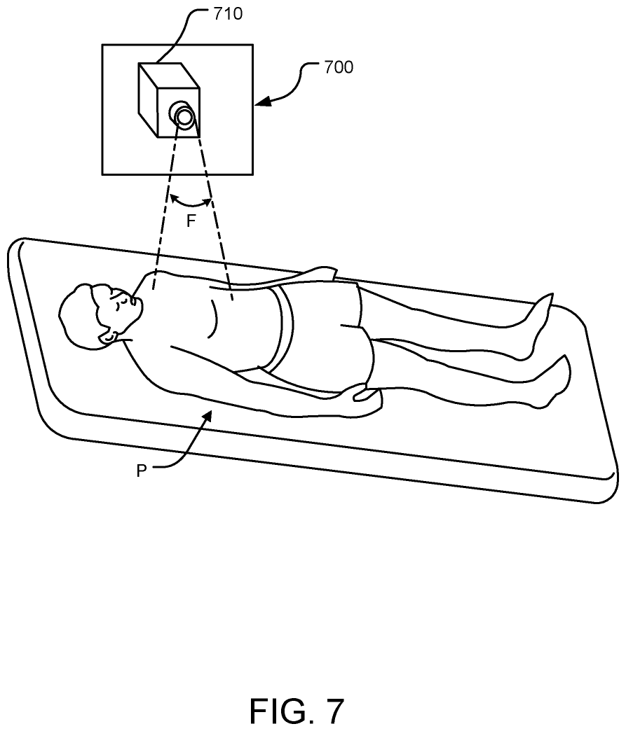

FIG. 7 shows a scenario where a patient P is being monitored by a non-contact system 700 for respiratory rate and tidal volume, for example, indicative of hyperventilation, a symptom of hyperglycemia.

As with the other systems, the system 700 may be any of the systems 100, 200, or other described above, or variations thereof. The system 700 has a camera system 710 with a field of view F that corresponds to an ROI on the patient P's chest. The camera system 710 can be any camera system (e.g., the camera system 114 or the cameras 214, 215). The camera system 710 monitors the ROI and the system 700 determines and records a signal or signal pattern, as a waveform, that can be used to identify hyperventilation. In other embodiments, the ROI may additionally or alternately encompass the patient P's facial region or mouth, monitoring for heavy mouth breathing.

Although not shown, another non-contact monitoring system may be arranged to monitor a patient P's heartrate, via the patient P's pulse; increased heartrate is a symptom of both hyperglycemia and hypoglycemia, as well as other diseases and conditions.

The non-contact monitoring systems described herein, and variations thereof, can be used in numerous methods to diagnose and evaluate certain diseases and conditions that have motion-based symptoms. The diagnosis can be used to determine a treatment for the patient.

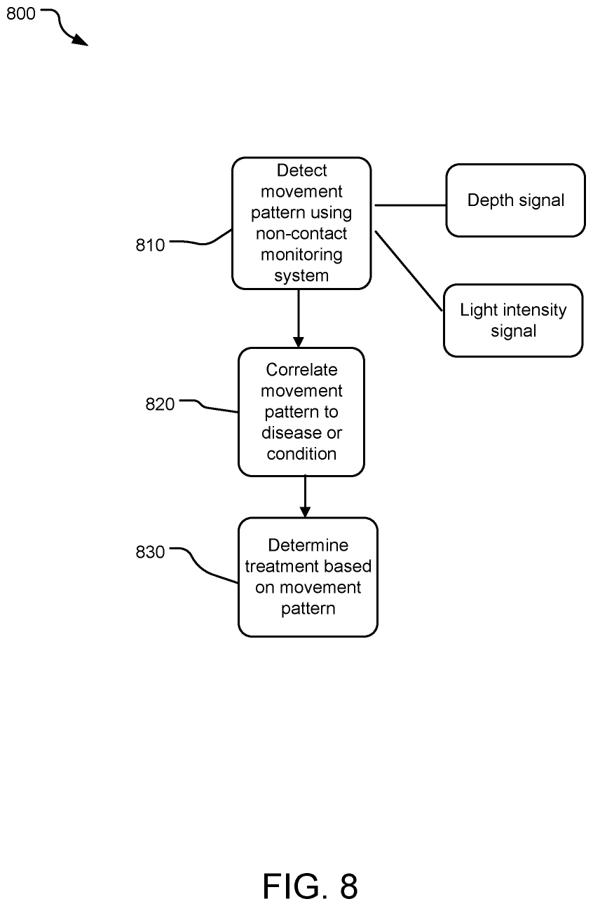

FIG. 8 shows a method 800 for determining possible presence of a disease and subsequent treatment for the disease with motion-based symptoms according to various embodiments described herein.

In a first step 810, a non-contact patient monitoring system is used to detect movement of a patient, e.g., a pattern of movement. The movement may be detected using one or both of a depth signal and a light intensity signal of reflected (projected) images. The movement or movement pattern is correlated to a disease, such as, e.g., Essential tremor or Parkinson's disease, in step 820. The movement may be, e.g., repeated leg spasms. From the movement or movement pattern, a treatment or therapy (e.g., medication, dosage of medication, frequency of medication, neurological stimulus, etc.) is determined in step 830 for the patient.

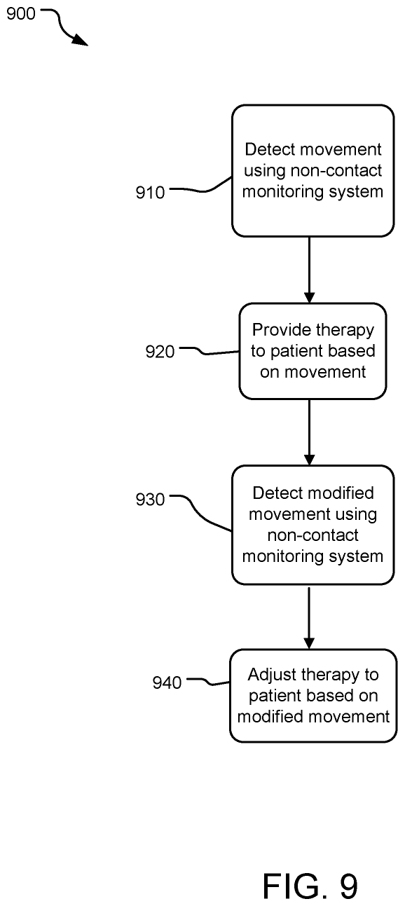

FIG. 9 shows another method, this method 900 for determining and adjusting treatment for a disease with motion-based symptoms according to various embodiments described herein.

In a first step 910, a non-contact patient monitoring system is used to detect movement of a patient, e.g., a pattern of movement. A therapy or treatment (e.g., neurological stimulus, medication, etc.) is provided to the patient in step 920 based on the detected movement; the therapy or treatment provided may be an adjustment to an already existing therapy or treatment or may be new. The non-contact monitoring system is again used in step 930 to detect movement of the patient, the movement modified based on after the therapy or treatment has been administered. This detection step 930 may be done immediately after or almost simultaneously with step 920 (e.g., within one hour), or this detection step 930 may be done at a later time and/or date, e.g., within one day, e.g., in a week. From this second detection step 930, the patient's caretaker can see the effects of the therapy or treatment. From the second monitoring, based on the modified movement detected in step 930, the therapy or treatment to the patient is adjusted in step 940.

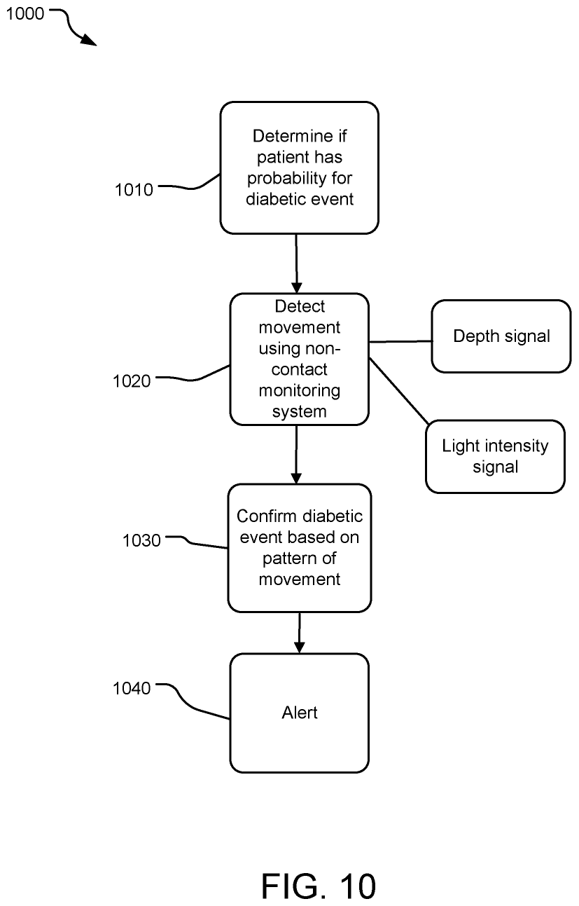

FIG. 10 shows an example method 1000 for determining when a patient has a hypoglycemic or hyperglycemic event or episode, according to various embodiments described herein.

In a first step 1010, it is determined if a patient has a probability or potential for hypoglycemia or hyperglycemia, both indicators of diabetes. If the probability is sufficiently high or another reason warrants monitoring, the patient P is monitored by a non-contact patient monitoring system such as described herein.

In a second step 1020, the non-contact patient monitoring system is used to detect movement of the patient, e.g., a pattern of movement, that may be indicative of a hypoglycemic or hyperglycemic event or episode. For example, if the patient has a tendency for hypoglycemia, the patient can be monitored for general unrest or restlessness; if the patient has a tendency for hyperglycemia, the patient can be monitored for hyperventilation or increased respiratory rate. The movement may be detected using one or both of a depth signal and a light intensity signal of reflected (projected) images.

In step 1030, the movement is further monitored to determine if a pattern of the movement is indicative of a diabetic event or episode. For example, general unrest or restlessness related to hypoglycemia increases over time; hyperventilation or increased respiratory rate related to hyperglycemia also increases over time. If a diabetic event or episode is determined, an alert is issued in step 1040.

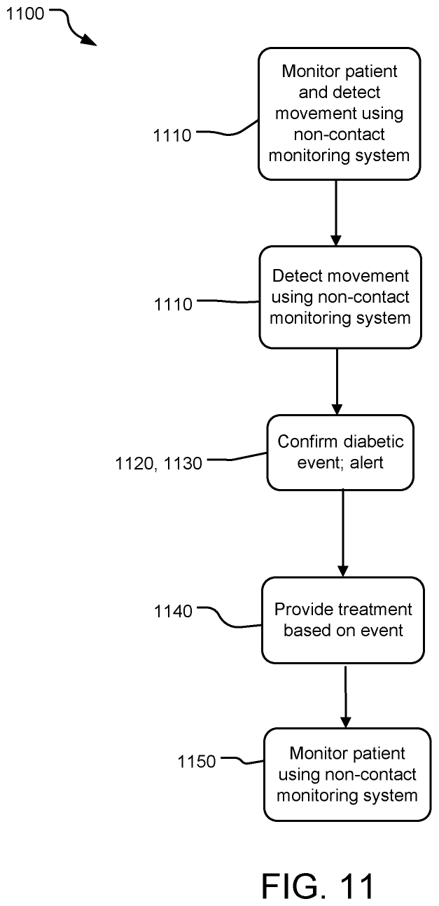

FIG. 11 shows another example method, this method 1100 for treatment of a patient having a hypoglycemic or hyperglycemic event or episode, according to various embodiments described herein.

In a first step 1110, (after it is determined that a patient has a probability or potential for hypoglycemia or hyperglycemia, both indicators of diabetes, that is sufficiently high to warrant monitoring), a non-contact patient monitoring system is used to detect movement of a patient. After confirmation that the movement represents a diabetic event or episode, in step 1120, an alert is issued is step 1130.

A treatment (e.g., insulin or sugar) is provided to the patient in step 1140 based on the determined diabetic event or episode.

The non-contact monitoring system is again used in step 1150 to monitor the patient after the treatment has been administered. This monitoring step 1150 may be done immediately after or almost simultaneously with step 1140 (e.g., within ten minutes, or within one hour). From this second monitoring step 1150, the patient's caretaker can see the effects of the treatment.

The above specification and examples provide a complete description of the structure and use of exemplary embodiments of the invention. The above description provides specific embodiments. It is to be understood that other embodiments are contemplated and may be made without departing from the scope or spirit of the present disclosure. The above detailed description, therefore, is not to be taken in a limiting sense. For example, elements or features of one example, embodiment or implementation may be applied to any other example, embodiment or implementation described herein to the extent such contents do not conflict. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about,” whether or not the term “about” is immediately present. Accordingly, unless indicated to the contrary, the numerical parameters set forth are approximations that can vary depending upon the desired properties sought to be obtained by those skilled in the art utilizing the teachings disclosed herein.

As used herein, the singular forms “a”, “an”, and “the” encompass implementations having plural referents, unless the content clearly dictates otherwise. As used in this specification and the appended claims, the term “or” is generally employed in its sense including “and/or” unless the content clearly dictates otherwise.

1. A non-contact monitoring system for monitoring a patient, the system comprising:
a depth-sensing camera;
a display; and
a processor operably connected to a memory of the system, the processor configured to implement steps to:
detect a pattern of movement of the patient in a region of interest (ROI) with the non-contact monitoring system based on a change in a depth signal over time;
correlate the detected pattern of movement to a disease or condition; and
determine a treatment for the patient for the disease or condition.

2. The system of claim 1, wherein the processor is further configured to detect movement of the patient by a change in a light intensity signal over time.

3. The system of claim 2, wherein the system further comprises a projector and the processor is further configured to detect movement of the patient from a light intensity reflection signal of a feature projected onto the patient in the ROI.

4. The system of claim 1, wherein the processor is further configured to notify a user via a display of the determined treatment for the patient for the detected disease or condition.

5. The system of claim 1, wherein the processor is configured to correlate the detected pattern of movement to one or more of Essential tremor, Parkinson's disease, restless leg syndrome, and a diabetic episode.

6. A method of determining a treatment for a disease or condition, the method comprising:
detecting movement of a patient in a region of interest (ROI) with a non-contact monitoring system based on a change in a depth signal over time;
correlating the detected movement to a disease or condition; and
determining a treatment for the patient for the disease or condition.

7. The method of claim 6, wherein the patient has a sufficient probability for an occurrence of a diabetic event, and confirming the detected movement is correlated to a diabetic event.

8. The method of claim 7, further comprising:
dependent on confirming the movement is related to the diabetic event, determining a treatment for the patient for the diabetic event.

9. The method of claim 6, wherein detecting movement of the patient in the ROI comprises monitoring one or more of a leg region of the patient, a torso region of the patient, and a facial region of the patient.

10. The method of claim 9, wherein correlating the detected movement to a disease or condition comprises correlating the detected movement to one of Essential tremor, Parkinson's disease, and restless leg syndrome.

11. A method comprising:
detecting movement of a patient in a region of interest (ROI) with a non-contact monitoring system with a depth-based signal over time;
providing a therapy to the patient based on the detected movement;
after providing the therapy, detecting modified movement of the patient in the ROI with the non-contact monitoring system; and
adjusting the therapy to the patient based on the detected modified movement.

12. The method of claim 11, wherein adjusting the therapy comprises adjusting a neurostimulator implanted in the patient.

13. The method of claim 11, wherein detecting movement of the patient in the ROI and detecting modified movement in the ROI comprises monitoring one or more of a leg region of the patient, a torso region of the patient, and a facial region of the patient.

14. The method of claim 11, wherein the movement of the patient is further detected by a change in a light intensity signal over time.

15. The method of claim 14, wherein the light intensity signal is a reflection of a feature projected onto the patient in the ROI.
